(19) United States
(12) Patent Application Publication
(10) Pub. No.: US 2024/0230654 A1
(43) Pub. Date: Jul. 11, 2024

(54) SYSTEMS AND METHODS FOR IDENTIFYING CANCEROUS TISSUE

(71) Applicant: Caleb Ericson, Geneva, IL (US)

(72) Inventor: Caleb Ericson, Geneva, IL (US)

(21) Appl. No.: 18/404,819

(22) Filed: Jan. 4, 2024

Related U.S. Application Data
(60) Provisional application No. 63/576,426, filed on Jan. 5, 2023.

(51) Int. Cl. G01N 33/574; G01N 1/30; G01N 1/31; G01N 33/543

(52) U.S. Cl. CPC G01N 33/57484; G01N 1/30; G01N 1/312; G01N 33/54386; B01L 2200/16; B01L 2300/0654; G01N 2001/302; G01N 2001/315

(57) ABSTRACT

A surgical system for identifying cancerous tissue includes a dish configured to receive a tissue sample, a frame including a base defining a recess configured to receive the dish, an upright extending upward from the base, and an overhang extending over the base, and a light source coupled to the overhang and positioned to emit light toward the recess.

### CROSS-REFERENCE TO RELATED PATENT APPLICATION

This application claims the benefit of and priority to U.S. Provisional Application No. 63/576,426, filed on Jan. 5, 2023, the entire disclosure of which is hereby incorporated by reference herein.

### BACKGROUND

The present disclosure relates generally to identification of cancerous tissue. More specifically, the present disclosure relates to identification of skin cancer, such as melanoma or carcinoma.

Some treatments for skin cancer involve surgically removing sections of tissue that have been identified as containing cancerous tissue. While the general area containing the cancerous tissue can be marked for the surgeon, it can be difficult for the surgeon to know the exact location and boundaries of the cancerous tissue. It is desirable to minimize the amount of tissue that is removed to mitigate the negative impact of the surgery on the patient. However, if the section of tissue removed is insufficient and some of the cancerous tissue is not removed, the treatment may be ineffective.

### SUMMARY

At least one embodiment relates to a surgical system for identifying cancerous tissue. The surgical system includes a dish configured to receive a tissue sample, a frame including a base defining a recess configured to receive the dish, an upright extending upward from the base, and an overhang extending over the base, and a light source coupled to the overhang and positioned to emit light toward the recess.

Another embodiment relates to a surgical method including removing tissue from a patient, the tissue containing cancerous cells and having a margin that is exposed when the tissue is removed from the patient, bathing the tissue in a cancer identifying fluid that is configured to emit light in response to the cancer identifying fluid both (a) contacting the cancerous cells and (b) being exposed to an activation light, exposing the cancer identifying fluid to the activation light, and determining if the cancerous cells are exposed along the margin based on whether or not the light is emitted by the cancer identifying fluid.

Another embodiment relates to a surgical system for identifying cancerous tissue. The surgical system includes a dish configured to receive a tissue sample, a frame, a light source positioned to emit ultraviolet light downward toward the dish, a battery coupled to the frame and configured to supply electrical energy to the light source, and a container of cancer identifying fluid coupled to the frame. The frame includes a first portion defining a recess configured to receive the dish, a second portion extending above the first portion, and a third portion extending between the first portion and the second portion and fixedly coupling the first portion to the second portion. The first portion, the second portion, and the third portion define a passage. The light source is coupled to the third portion. A wire extends through the passage and electrically couples the battery and the light source. The cancer identifying fluid is configured to emit visible light in response to the cancer identifying fluid both (a) contacting cancerous tissue and (b) being exposed to the ultraviolet light.

This summary is illustrative only and is not intended to be in any way limiting. Other aspects, inventive features, and advantages of the devices or processes described herein will become apparent in the detailed description set forth herein, taken in conjunction with the accompanying figures, wherein like reference numerals refer to like elements.

### BRIEF DESCRIPTION OF THE FIGURES

The disclosure will become more fully understood from the following detailed description, taken in conjunction with the accompanying figures, wherein like reference numerals refer to like elements, in which:

FIG. 1 is a block diagram of a surgical method, according to an exemplary embodiment.

FIG. 2 is a front view of an area of skin containing cancerous tissue, according to an exemplary embodiment.

FIG. 3 is a front view of an area of skin containing cancerous tissue, according to another exemplary embodiment.

FIG. 4 is a diagram illustrating an interaction between a cancer identifying fluid and cancerous tissue, according to an exemplary embodiment.

FIG. 5 is a graph illustrating the concentration of cancer identifying fluid at various depths within a tissue sample after bathing the tissue sample for various lengths of time, according to an exemplary embodiment.

FIG. 6 is a perspective view of an inspection assembly for use with the surgical method of FIG. 1, according to an exemplary embodiment.

FIG. 7 is a right side view of the inspection assembly of FIG. 6.

FIG. 8 is a top view of the inspection assembly of FIG. 6.

FIG. 9 is a front view of the inspection assembly of FIG. 6.

FIG. 10 is a right section view of the inspection assembly of FIG. 6.

### DETAILED DESCRIPTION

Before turning to the figures, which illustrate certain exemplary embodiments in detail, it should be understood that the present disclosure is not limited to the details or methodology set forth in the description or illustrated in the figures. It should also be understood that the terminology used herein is for the purpose of description only and should not be regarded as limiting.

Referring generally to the figures, a system and method for identifying and removing cancerous tissue are shown according to an exemplary embodiment. The system and method facilitate confirming whether all cancerous tissue has been removed during a surgical procedure or if additional surgery is needed. When a section of tissue known to contain cancerous tissue is excised (e.g., cut away and removed), the edge that is cut away forms a margin of the removed tissue. If the margin exposes cancerous tissue, the margin is considered a positive margin. If the margin does not expose cancerous tissue (e.g., exposes only healthy tissue), the margin is considered a negative margin. The presence of a positive margin means that the cut passed through the cancerous tissue, and a portion of the cancerous tissue remains in the patient.

In the method, a section of tissue known to contain cancerous tissue is excised from a patient. The removed tissue is placed in a dish of an inspection assembly and bathed with a cancer identifying fluid for a threshold period. In some embodiments, each piece of excised tissue is exposed to the cancer identifying fluid for ten minutes. The cancer identifying fluid includes antibodies that bond to cancerous tissue. Accordingly, the antibodies will bond to positive margins but will not bond to negative margins. In some embodiments, the cancer identifying fluid would be cetuximab bonded to tryptophan. In other embodiments, the cancer identifying fluid includes another combination of an antibody and a fluorescent tag.

The antibodies are tagged with a molecule that gives off visible light when exposed to an activating light (e.g., ultraviolet light). When the removed tissue is exposed to the activating light, the positive margins give off visible light, while the negative margins do not give off visible light. Accordingly, a user can visually identify the size, shape, and location of any positive margins based on the presence or absence of visible light. If the user can see any visible light being emitted from the removed tissue, they may determine that additional cancerous tissue is still present in the patient and that additional tissue should be removed. The systems and methods described herein facilitate rapid identification of positive margins during the surgical procedure, facilitating identification and removal of all of the cancerous tissue in one sitting (e.g., without ending and subsequently restarting the surgical procedure).

### Surgical Method

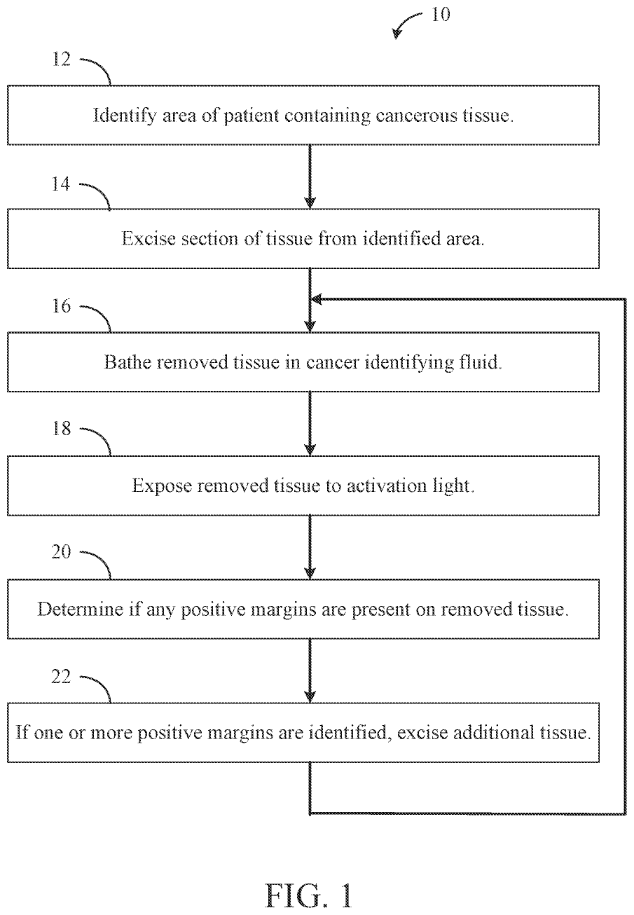

Referring to FIG. 1, a surgical method for identifying and removing cancerous tissue (e.g., tissue containing cancerous cells) is shown as method 10 according to an exemplary embodiment. The method 10 may be utilized to treat skin cancer, such as melanoma or carcinoma. The method 10 permits a surgeon to remove cancerous tissue from a patient and subsequently confirm that all of the cancerous tissue has been removed. Accordingly, the method 10 increases the efficacy of cancer treatment by preventing a situation in which a portion of the cancerous tissue remains after the surgery and continues to harm the patient.

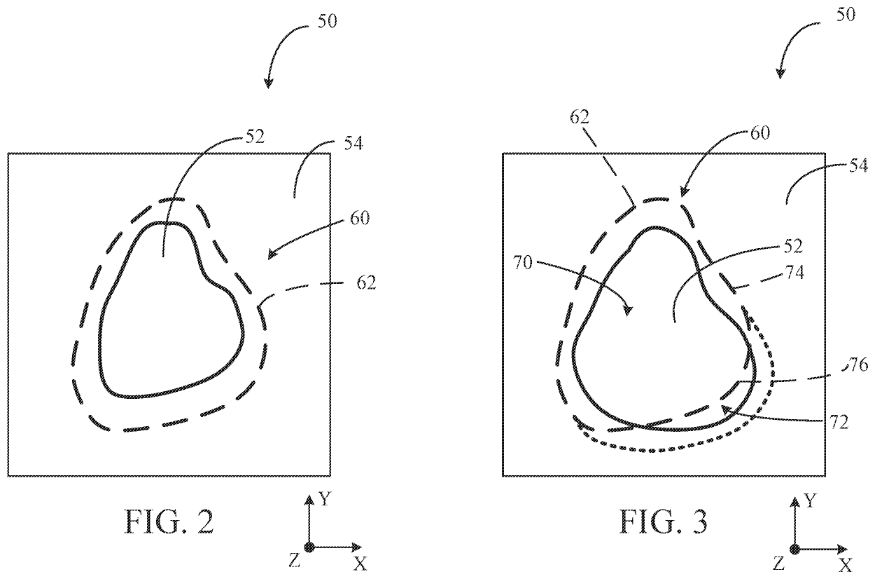

In step 12 of the method 10, an area of a patient containing cancerous tissue is identified. An example of such an area is shown in FIGS. 2 and 3 as area 50 containing cancerous tissue 52. As shown, the cancerous tissue 52 is surrounded by healthy tissue 54. The area 50 may include the skin of the patient, and thus may be positioned along the exterior of the patient's body. In some embodiments, the area 50 is larger than the cancerous tissue 52, such that the area 50 also includes the healthy tissue 54.

To facilitate description, a coordinate system including a lateral direction X, a vertical direction Y, and a depth direction Z is defined for FIGS. 2 and 3. In FIGS. 2 and 3, the skin of the patient extends parallel to the image (i.e., in an XY plane containing the lateral direction X and the vertical direction Y). The depth direction Z extends into and out of the skin of the patient.

The area 50 may be identified by one or more individuals (e.g., the patient, a doctor, a surgeon, a surgical assistant, a nurse, etc.) utilizing one or more techniques (e.g., visually, through a biopsy, based on an identifying marking made by another individual, etc.). The patient may initially visually identify the area 50 based on one or more visual characteristics of the area 50. By way of example, the area 50 may include a visible growth or marking, such as a mole, spot, sore, or textured (e.g., scaly) patch of skin. The patient may determine that the area 50 potentially contains cancerous tissue based on the shape, size, color, or texture of the visible growth. A doctor (e.g., a dermatologist) may identify the area 50 (e.g., visually, at the direction of the patient, etc.). The doctor may confirm that the area 50 contains the cancerous tissue 52 by excising (e.g., surgically removing) a portion of the tissue from the area 50 and performing a biopsy. If the area 50 has been confirmed to contain the cancerous tissue 52, the area 50 may be marked with a visual indicator (e.g., an ink-based surgical marker or tattoo). A surgeon may again identify the area 50 prior to performing surgery based on the visible growth or the visible indicator.

In step 14 of the method 10, a section of tissue is excised from the identified area. As shown in FIGS. 2 and 3, a section 60 of the tissue contained within the area 50 is excised. The section 60 has an edge, perimeter, outer surface, or border, shown as cut surface 62. A surgeon cuts into the skin of the patient to separate the section 60 and form the cut surface 62. The surgeon may utilize a cutting tool, such as a scalpel, to cut the section 60 away from the surrounding tissue. The cut surface 62 may extend in the depth direction Z to permit fully separating the section 60 from the surrounding tissue. The surgeon may then pull or pry the section 60 away from the surrounding tissue to remove the section 60 from the patient.

The cut surface 62 may be made up of one or more edges, perimeters, surfaces, or borders, referred to herein as margins. A margin may be identified as a positive margin or as a negative margin. A margin may be considered a negative margin if no cancerous tissue (e.g., cancer cells) is detected along the margin. A margin may be considered a positive margin if some cancerous tissue is detected along the margin. The cut surface 62 may include only negative margins (e.g., if no cancerous tissue is detected along any portion of the cut surface 62), only positive margins (e.g., if cancerous material is detected along the entirety of the cut surface 62), or both positive and negative margins (e.g., if cancerous material is detected along only a portion of the cut surface 62). Some margins of the cut surface 62 may not be visible in FIGS. 2 and 3, as the cut surface 62 extends in the depth direction Z.

In the example shown in FIG. 2, the cut surface 62 surrounds the cancerous tissue 52, and none of the cancerous tissue 52 extends beyond the cut surface 62. Accordingly, the cut surface 62 is positioned such that all of the cancerous tissue 52 will be removed when the section 60 is removed from the patient. As the cut surface 62 does not intersect the cancerous tissue 52, the cut surface 62 does not include any positive margins (i.e., the cut surface 62 includes only negative margins). As such, if no positive margins can be detected on the cut surface 62, it can be inferred that all of the cancerous tissue 52 has been removed and no further surgical procedures are required to eliminate further cancerous tissue 52.

In the example shown in FIG. 3, the cut surface 62 surrounds only a first portion 70 of the cancerous tissue 52, and a second portion 72 of the cancerous tissue 52 extends beyond the cut surface 62. Accordingly, the cut surface 62 intersects the cancerous tissue 52, and only the first portion 70 of the cancerous tissue 52 will be removed when the section 60 is removed from the patient. The portion of the cut surface 62 that does not intersect the cancerous tissue 52 forms negative margins 74 between the first portion 70 and the surrounding healthy tissue 54. The portion of the cut surface 62 that intersects the cancerous tissue 52 forms a positive margin 76 that extends between the first portion 70 and the second portion 72. As such, if one or more positive margins can be detected on the cut surface 62, it can be inferred that a portion of the cancerous tissue 52 remains in the patient and further surgical procedures are required.

In step 16 of the method 10, the removed tissue (e.g., the section 60) is bathed in a cancer identifying fluid (e.g., a cancer identification solution, a cancer identifying solution, etc.). By bathing (e.g., soaking, immersing, covering, etc.) the removed tissue in the cancer identifying fluid, the entire outer surface of the removed tissue is exposed to the cancer identifying fluid. The removed tissue may be bathed in the cancer identifying fluid for at least a threshold period of time (e.g., 10 minutes) to permit the cancer identifying fluid to penetrate the removed tissue and/or interact with the outer surface of the removed tissue. After the threshold period of time has elapsed, step 18 of the method 10 is initiated, in which the removed tissue (e.g., the section 60) is exposed to an activation light.

The cancer identifying fluid is a fluid (e.g., a liquid) that provides an indication (e.g., a visible indication) when in contact with a cancerous tissue. The cancer identifying fluid may emit visible light only when both (a) the cancer identifying fluid is in contact with cancerous tissue and (b) the cancer identifying fluid is exposed to an activation light of a given wavelength or range of wavelengths. As the entire outer surface of the removed tissue is exposed to the cancer identifying fluid in step 16, any positive margins on the outer surface of the removed tissue will have been exposed to the cancer identifying fluid when step 18 is initiated. These positive margins will then emit visible light when exposed to the activation light in step 18, identifying the locations of any positive margins.

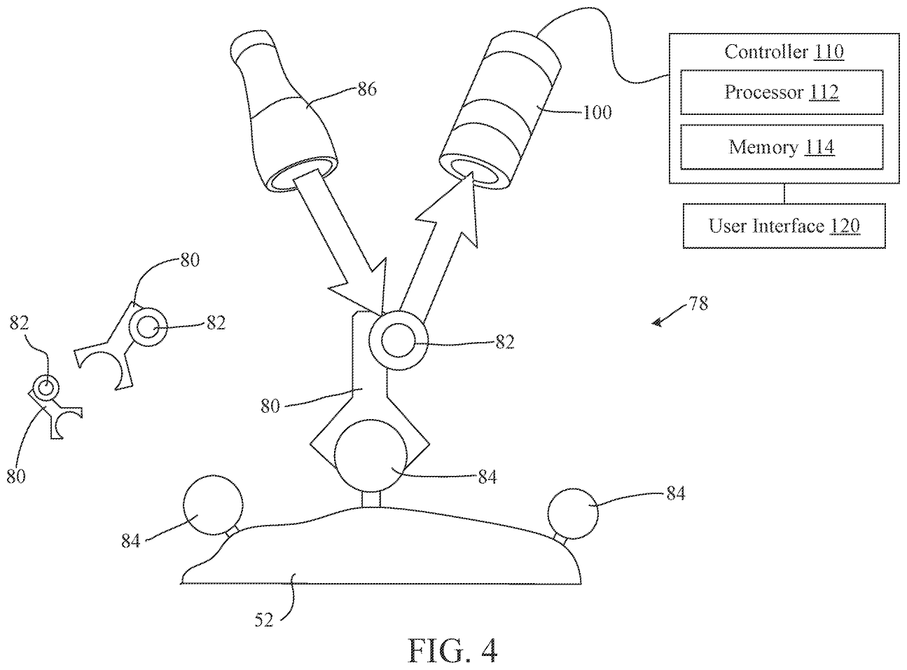

In some embodiments, the cancer identifying fluid contains epidermal growth factor receptor (EGFR) antibodies that are each tagged with (e.g., coupled to) a fluorescent molecule. Referring to FIG. 4, a cancer identifying fluid 78 contains EGFR antibodies 80 tagged with fluorescent molecules, shown as molecules 82. The molecules 82 emit visible light when exposed to light in the ultraviolet range (e.g., light having a wavelength between 100 nm and 400 nm). In other embodiments, the molecules 82 emit light that is not visible (e.g., infrared light having a wavelength between 700 nm and 1 mm, etc.) when exposed to light in the ultraviolet range. The emitted light may be detected by a sensor (e.g., the camera 100) instead of a user's eyes directly. In some embodiments, the cancer identifying fluid is Cetuximab tagged with tryptophan using heat-based reactions. In other embodiments, the method 10 utilizes a different type of cancer identifying fluid. By way of example, the antibodies 80 may include HMB45 when detecting melanoma. By way of another example, the antibodies 80 may include MUC4 when detecting carcinoma.

Cancerous tissues contain overexpressed EGFR proteins, which trigger the reproduction of cells. The cancerous tissue 52 is shown in FIG. 4 as including overexpressed EGFR proteins, shown as EGFR proteins 84. When the cancerous tissue 52 comes into contact with the cancer identifying fluid 78, the EGFR antibodies 80 attack the overly expressed EGFR proteins 84, bonding the EGFR antibodies 80 to the EGFR proteins 84. A light source 86 (e.g., a light-emitting diode) emits ultraviolet light (e.g., activation light) toward the cancerous tissue 52, and the ultraviolet light is received by the molecules 82 of the bonded EGFR antibodies 80. This ultraviolet light is outside of the visible spectrum and thus cannot be detected by the human eye. In response to receiving the ultraviolet light, the molecules 82 of the bonded EGFR antibodies 80 emit visible light (e.g., light having a wavelength between 400 nm and 700 nm) that can be detected by the human eye.

Accordingly, when the section 60 has one or more positive margins, the cancerous tissue 52 of the positive margins is exposed to the cancer identifying fluid 78, and the EGFR antibodies 80 bond to the cancerous tissue 52. Upon exposure to the activation light (e.g., ultraviolet light from the light source 86), the molecules 82 along the positive margins will glow, indicating the size, shape, and location of each positive margin.

When the section 60 does not have any positive margins (i.e., the section 60 has only negative margins), the cancer identifying fluid 78 may soak through the healthy tissue 54 to reach the cancerous tissue 52, but the healthy tissue 54 may block the cancer identifying fluid 78 from receiving the activation light from the light source 86. Alternatively, the healthy tissue 54 may block the cancer identifying fluid 78 from reaching the cancerous tissue 52, preventing the EGFR antibodies 80 from bonding with the EGFR proteins 84. In either situation, upon exposure of the section 60 to the activation light, the section 60 will not emit any visible light.

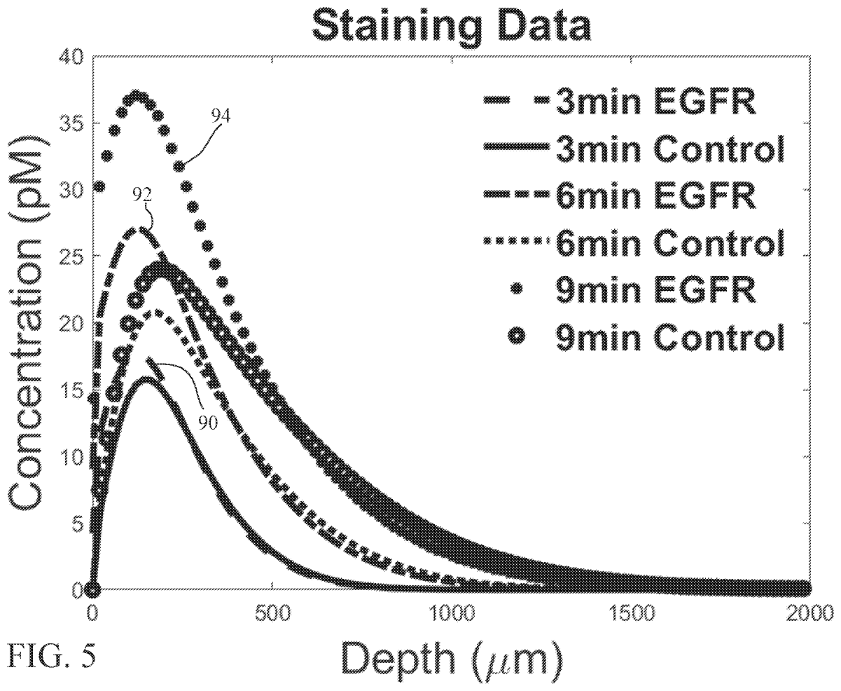

The threshold length of time for which the removed tissue is bathed in the cancer identifying fluid may be predetermined based upon the amount of time required for the cancer identifying fluid to penetrate the surface of the removed tissue. FIG. 5 is a chart illustrating the change in concentration of the cancer identifying fluid throughout the depth of the removed tissue for different bathing times. The data of FIG. 5 was determined experimentally. As shown, FIG. 5 includes a first data series 90, a second data series 92, and a third data series 94. The first data series 90 illustrates the concentration of the cancer identifying fluid throughout the depth of a removed tissue sample that was bathed in the cancer identifying fluid for three minutes. The second data series 92 illustrates the concentration of the cancer identifying fluid throughout the depth of a removed tissue sample that was bathed in the cancer identifying fluid for six minutes. The third data series 94 illustrates the concentration of the cancer identifying fluid throughout the depth of a removed tissue sample that was bathed in the cancer identifying fluid for nine minutes. As shown, increasing the length of time that the removed tissue is bathed results in (a) a significant increase in the peak concentration of the cancer identifying fluid and (b) a minor increase in the depth where the peak concentration is achieved.

In some embodiments, the threshold length of time is 10 minutes, such that the removed tissue is bathed for at least 10 minutes in step 16. Based on the data of FIG. 5, bathing the removed tissue for at least 10 minutes ensures that the concentration of the cancer identifying fluid is sufficient to be visible if the removed tissue has a positive margin. In other embodiments, the threshold length of time is decreased (e.g., to 9 minutes, to 6 minutes, etc.). Decreasing the threshold length of time reduces the amount of time required to perform the method 10, but the concentration of the cancer identifying fluid may not be sufficient to produce light that can be identified by the surgeon.

Referring again to FIG. 1, in step 20 of the method 10, it is determined if any positive margins are present on the removed tissue (e.g., the section 60). In some embodiments, this determination is made by the surgeon or another individual that is involved in the analysis of the removed tissue (e.g., a technician, a nurse, etc.). By way of example, a surgeon may visually inspect the exterior of the removed tissue while the removed tissue is being exposed to the activation light. The surgeon may rotate or otherwise manipulate the removed tissue to ensure that all surfaces of the removed tissue are exposed to the activation light and inspected.

If the surgeon cannot detect any visible light emitting from the removed tissue, the surgeon may conclude that no positive margins are present. Accordingly, the surgeon may conclude that all of the cancerous tissue has been removed and that the surgery can conclude. The surgeon may then dress (e.g., clean and stitch) the wound left by the removal of the tissue from the patient and conclude the surgery.

If the surgeon detects visible light emitting from the removed tissue, the surgeon may conclude that one or more positive margins are present. Accordingly, the surgeon may conclude that a portion (e.g., the second portion 72) of the cancerous tissue is still present in the patient and that further surgery is required. As part of the inspection, the surgeon may take note of (a) the quantity of positive margins on the removed tissue, (b) the size of the positive margins, and/or (c) the locations of the positive margins. This information may facilitate later portions of the method 10.

In step 22 of the method 10, additional tissue is excised from the patient if one or more positive margins were identified in step 20. In the example shown in FIG. 3, the first portion 70 of the cancerous tissue 52 is removed in step 14. The goal of step 22 is therefore to remove the second portion 72 to eliminate all of the cancerous tissue 52 from the patient. To accomplish this, a section 96 of the tissue contained within the area 50 is excised. The section 96 has an edge, perimeter, outer surface, or border, shown as cut surface 98. A surgeon cuts into the skin of the patient to separate the section 60 and form the cut surface 98. The surgeon may then pull or pry the section 96 away from the surrounding tissue to remove the section 96 from the patient.

It is desirable for the cut section 96 to include the second portion 72 of the cancerous tissue 52. In order to accomplish this, the surgeon may utilize the information gathered in step 20 when determining where to place the cut that forms the cut surface 98. Specifically, the surgeon may utilize (a) the quantity of positive margins on the section 60, (b) the size of the positive margins on the section 60, and/or (c) the locations of the positive margins on the section 60.

The quantity of positive margins on the section 60 indicates the number of additional sections (e.g., the second portion 72) of cancerous tissue 52 still left within the patient. By way of example, if the section 60 were to include two separate (e.g., not continuous) positive margins, the surgeon could conclude that there are still two sections of cancerous tissue 52 left within the patient. The surgeon could then determine how many additional cuts should be made based on this number. By way of example, if there were two positive margins, the surgeon could conclude that two additional cuts will be necessary.

The location of a positive margin on the section 60 indicates the location of the corresponding section of cancerous tissue 52 still left within the patient. Accordingly, the surgeon may ensure that the next section to be removed is at the same position as the positive margin. In order to accomplish this, the surgeon may have to determine the original orientation of the section 60 within the patient. By way of example, the surgeon may determine the original orientation of the section 60 based on markings (e.g., visual indicators) made on the section 60 and/or the surrounding tissue prior to removal of the section 60 (e.g., a V-shaped marking drawn across the section 60 and the surrounding tissue). In one such example, a surgeon uses a surgical marker to make a line across the section 60 and the surrounding tissue prior to excising the section 60. The surgeon may use this visual indicator to determine the original orientation of the section 60 prior to excision. By way of another example, the surgeon may determine the original orientation of the section 60 based on the shape of the section 60 and the corresponding recess in the surrounding tissue.

The size of a positive margin on the section 60 indicates the size of the corresponding section of cancerous tissue 52 still left within the patient. By way of example, the width and depth of the positive margin may be equal to the width and depth, respectively, of the cancerous tissue 52 at the cut surface 62. Accordingly, the surgeon may ensure that the next section to be removed is at least as wide and deep as the positive margin.

After the section 96 is removed, steps 16-20 may be repeated with the section 96. However, section 96 will have at least one positive margin along the surface that was previously exposed to the section 60. When determining if the section 96 has any positive margins in step 20, the at least one positive margin along the surface that was previously exposed to the section 60 will be ignored. If the section 96 is determined to have no other positive margins, the surgeon may determine that all of the second portion 72 of the cancerous tissue 52 has been removed, dress the patient's wound, and conclude the surgery. If one or more new positive margins are identified, then step 22 may be repeated.

Beneficially, the method 10 permits a surgeon to confirm that all cancerous material has been removed from a patient when treating skin cancer. Without the method 10, a surgeon could unknowingly leave behind a portion of the cancerous material, which could continue to harm the patient. The inspection steps of the method 10 (e.g., steps 16-20) can be performed quickly (e.g., in 10-20 minutes) and within the operating room where the patient is being held. This permits the surgeon to (a) remove a first section of tissue, (b) determine that additional cancerous tissue is still present within the patient, and (c) remove the additional cancerous tissue, all within a single surgery. Other surgical methods require a tissue sample to be sent to a separate lab for analysis. If this analysis determines that a portion of the cancerous tissue was not removed, the patient is required to return for a second surgery. Accordingly, the method 10 eliminates the cost and potential patient frustration associated with a second surgery.

In other embodiments, this determination of step 20 is at least partially automated. As shown in FIG. 4, a camera 100 is positioned to receive the visible light emitted by the molecules 82. The camera 100 may be configured to respond to receiving visible light. Accordingly, the camera 100 may detect whether or not the molecules 82 emit visible light. In other embodiments, the molecules 82 of the cancer identifying fluid emit a different wavelength of light that is not visible to the human eye (e.g., infrared light). In such embodiments, the camera 100 may be configured to respond to whatever type of light is emitted by the molecules 82.

The camera 100 is operatively coupled to a processing circuit, shown as controller 110. The controller 110 includes a processor 112 and a memory 114. The processor 112 may execute one or more instructions stored in the memory 114 to perform the functions described herein. The controller 110 may monitor a signal provided by the camera 100 and determine, based on the signal, if the removed tissue has any positive margins. By way of example, the controller 110 may utilize machine learning (e.g., neural networks) to perform image recognition or another type of analysis on one or more images captured by the camera and determine the presence, size, shape, and/or location of any positive margins. Based on whether or not the removed tissue has any positive margins, the controller 110 may determine if any additional cancerous tissue is still present within the patient. In some embodiments, the camera 110 includes a scintillation counter. The scintillation counter may detect radiation associated with the cancerous tissue (e.g., radiation emitted from the EGFR antibodies 80 and/or the molecules 82). The scintillation counter may accordingly be utilized to determine a quantity of cancer cells in the excised tissue and/or the surrounding tissue. The analysis of the scintillation counter may be utilized to determine a risk factor associated with not excising additional tissue.

The controller 110 is operatively coupled to an input device and/or output device, shown as user interface 120. The user interface 120 may include a display (e.g., a touchscreen display) for providing information to a user. By way of example, the user interface 120 may indicate whether or not any additional cancerous tissue is still present within the patient.

### Inspection Assembly

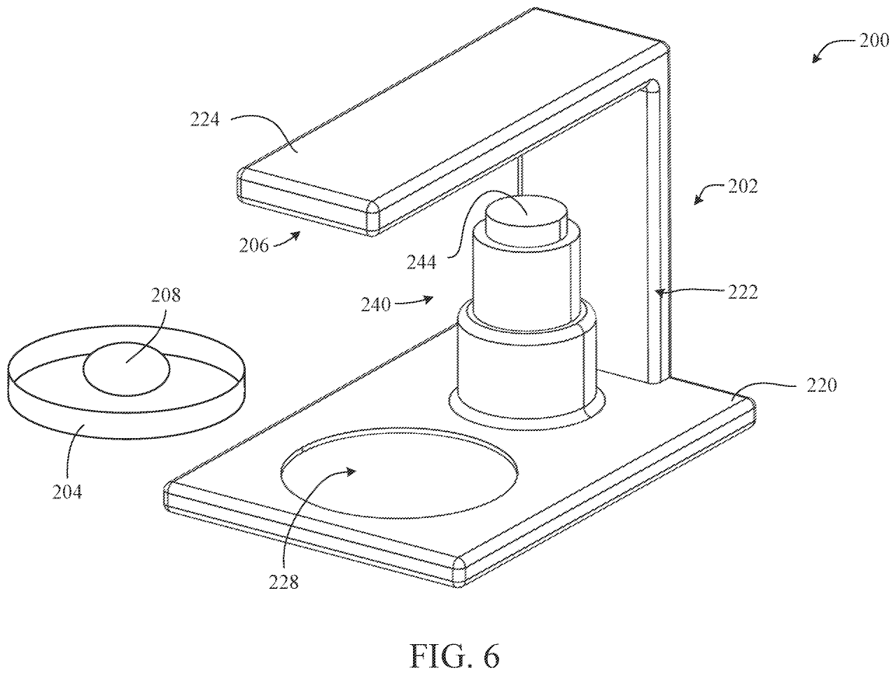

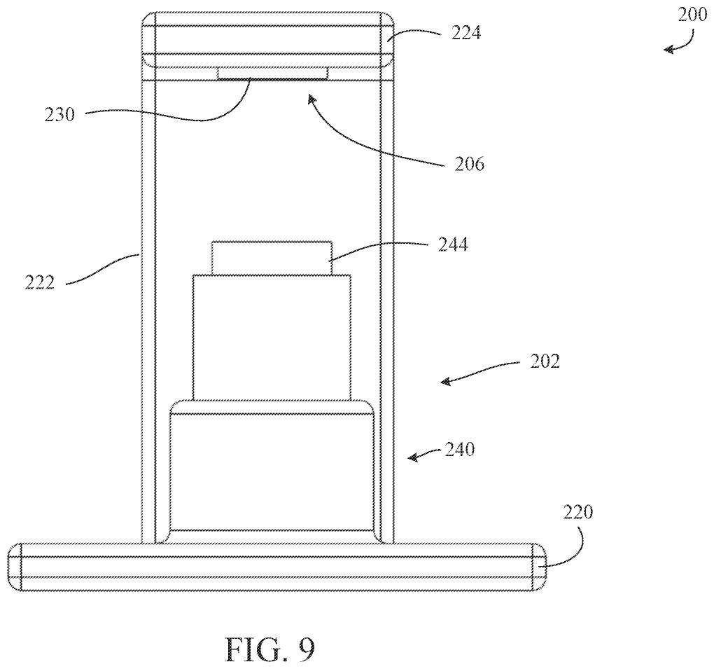

Referring to FIGS. 6-10, a surgical system, surgical assembly, clinical rig, or inspection system, is shown as inspection assembly 200, according to an exemplary embodiment. The inspection assembly 200 may facilitate performance of the method 10. By way of example, the inspection assembly 200 may be utilized by a surgeon, nurse, technician, or other personnel when performing steps 16-20 of the method 10.

The inspection assembly 200 includes a support structure, chassis, or fixture, shown as frame 202, a tray or container, shown as dish 204, and a light emission system, shown as activation system 206. The frame 202 supports the dish 204 and the activation system 206. The frame 202 may hold the activation system 206 in a predetermined and fixed relationship to the dish 204. The dish 204 supports and contains a sample of removed tissue (e.g., the section 60, the section 96, etc.), shown as tissue sample 208. The activation system 206 emits the activation light toward the sample to facilitate performance of the method 10.

The frame 202 includes a first section, lower section, base section, or plate, shown as base 220. The base 220 extends substantially horizontally and supports the other components of the frame 202. Extending upward from the base 220 is a riser, upright member, or upright section, shown as upright 222. The upright 222 is fixedly coupled to the base 220. The upright 222 is positioned near a rear end of the base 220. The upright 222 is approximately laterally centered relative to the base 220. A horizontal member, bar, upper section, or overhang section, shown as overhang 224, extends forward from the upright 222 such that the overhang 224 overhangs the base 220. The overhang 224 is fixedly coupled to an upper end portion of the upright 222. The overhang 224 is approximately laterally centered relative to the base 220 and the upright 222. The base 220, the upright 222, and the overhang 224 are fixedly coupled to one another. In some embodiments, the base 220, the upright 222, and the overhang 224 are integrally formed from a single, continuous piece of material (e.g., the frame 202 is 3D printed).

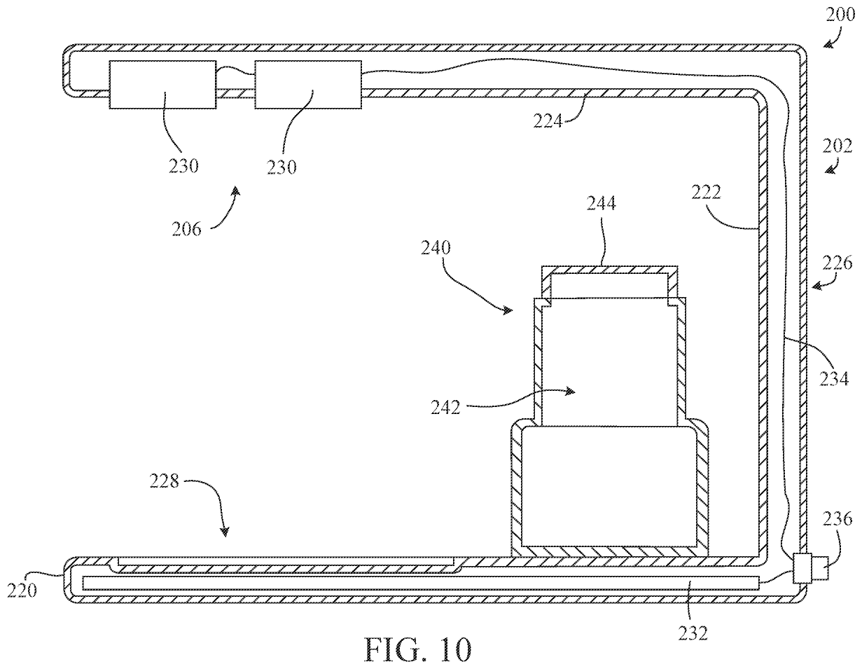

As shown in FIG. 10, the base 220, the upright 222, and the overhang 224 are all at least partially hollow, such that the frame 202 defines an internal volume 226 or passage. The internal volume 226 may be continuous throughout the base 220, the upright 222, and the overhang 224. The internal volume 226 may contain at least a portion of the activation system 206.

The base 220 defines an inset portion or recess, shown as dish recess 228. The dish recess 228 is inset from a top surface of the base 220 and extends vertically into the base 220. The dish recess 228 is shaped (e.g., circular) and sized to removably receive the dish 204. When the dish 204 is placed in alignment with the dish recess 228, gravity biases the dish 204 downward into the dish recess 228. When the dish 204 is fully seated within the dish recess 228, the base 220 holds the dish 204 in a fixed position relative to the frame 202 until a user lifts the dish 204 out of the dish recess 228.

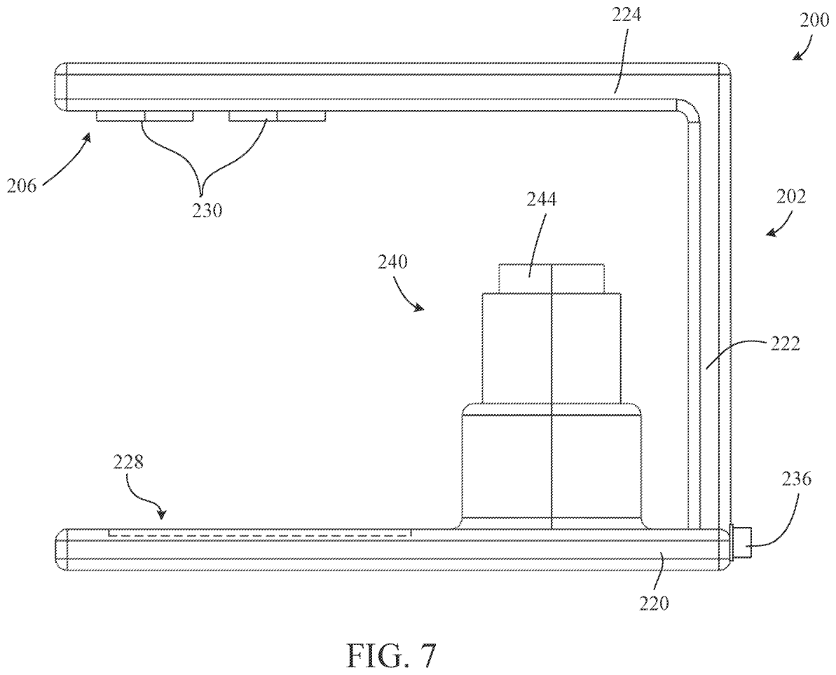

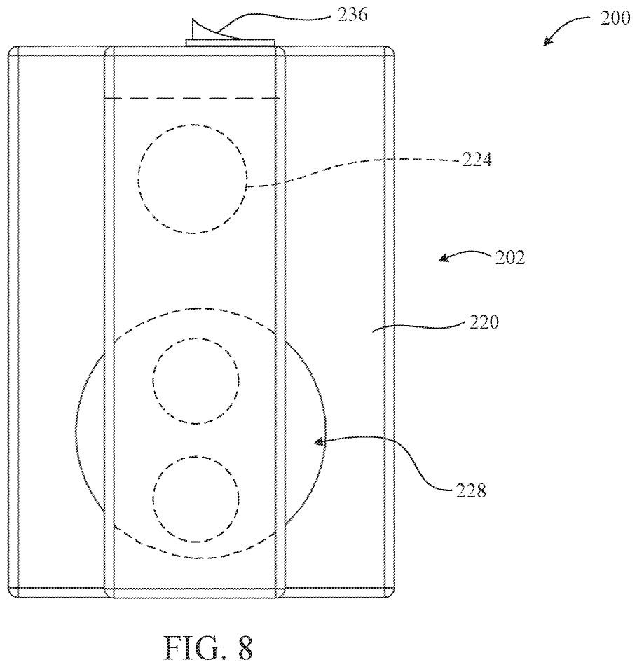

Referring to FIGS. 7 and 10, the activation system 206 includes a pair of light-emitting elements or light sources, shown as lights 230 (e.g., light-emitting diodes). The lights 230 emit light in response to receiving electrical energy. The light emitted by the lights 230 may be the activation light of step 18. Accordingly, the lights 230 may act as the light source 86. Each light 230 is coupled to the overhang 224 and oriented facing downward, toward the dish recess 228. The lights 230 are laterally centered about the overhang 224 and longitudinally offset from one another. The lights 230 may both be positioned directly above the dish recess 228. Accordingly, the activation light emitted by the lights 230 may be directed toward the dish recess 228.

The activation system 206 includes an electrical energy storage device (e.g., one or more batteries or capacitors, etc.), shown as battery pack 232, positioned within the internal volume 226 in the base 220. By placing the battery pack 232 in the base 220, the center of gravity of the inspection assembly 200 is lowered, and the stability of the inspection assembly 200 is increased. The battery pack 232 is configured to store electrical energy and deliver the stored electrical energy to power the lights 230. The battery pack 232 may be rechargeable or disposable. In embodiments where the battery pack 232 is rechargeable, the battery pack 232 may include an electrical connector positioned along the exterior of the frame 202, through which the battery pack 232 may be charged. In embodiments where the battery pack 232 is disposable, the frame 202 may include a removable door or panel that selectively permits removal and replacement of the battery pack 232. The battery pack 232 may utilize any type of battery technology (e.g., lithium-ion, lead-acid, nickel-cadmium, etc.).

The battery pack 232 is electrically coupled to the lights 230 through a series of conductors (e.g., wires, cables, etc.), shown as wires 234. The wires 234 extend through the internal volume 226, from the battery pack 232 to the lights 230. In some embodiments, the wires 234 electrically couple the lights 230 to the battery pack 232 in parallel.

A controller or electrical switch (e.g., a toggle switch), shown as switch 236, is electrically coupled to the wires 234 between the battery pack 232 and the lights 230. The switch 236 is reconfigurable (e.g., manually) between a connected configuration and a disconnected configuration. In the connected configuration, the switch 236 electrically couples the battery pack 232 to the lights 230 to activate the lights 230 (i.e., to turn the lights 230 on). In the disconnected configuration, the switch 236 electrically decouples the battery pack 232 from the lights 230 to deactivate the lights 230 (i.e., to turn the lights 230 off). The switch 236 may be manually reconfigured between the connected configuration and the disconnected configuration (e.g., by a user pressing on the switch 236). The switch 236 is fixedly coupled to a rear end of the frame 202. The switch 236 extends through the frame 202 to an exterior surface of the frame 202, such that the switch 236 can be accessed by a user positioned outside of the frame 202.

The inspection assembly 200 further includes a container of cancer identifying fluid (e.g., a bottle, a vessel, a can, a tube, a flask, a canister, a holder, etc.), shown as bottle 240. Storing the cancer identifying fluid in the bottle 240 may facilitate transporting the cancer identifying fluid with the inspection assembly 200. The bottle 240 defines an inner volume 242 that contains the cancer identifying fluid. The bottle 240 may include a cap 244 that can be selectively removed (e.g., by unscrewing, etc.) from the bottle 240 to permit the cancer identifying fluid to be added to or removed from the inner volume 242.

In some embodiments, the bottle 240 is removably coupled to the base 220. By way of example, the bottle 240 may be pressed into a recess defined by the base 220. By way of another example, the bottle 240 may be in threaded engagement with the base 220. By way of another example, the bottle 240 and/or the base 220 may include magnets to attract the bottle 240 to the base 220. The bottle 240 may be removed from the base 220 to pour the cancer identifying fluid into the dish 204.

In operation, the inspection assembly 200 may facilitate performing steps 16-20 of the method 10. After the tissue sample 208 has been removed from the patient, the tissue sample 208 is placed in the dish 204. The bottle 240 is removed from the base 220, the cap 244 is removed, and the cancer identifying fluid is poured into the dish 204, bathing the tissue sample 208. The tissue sample 208 bathes in the cancer identifying fluid until the threshold period of time has elapsed. At this time, the dish 204 is placed in the dish recess 228. A user engages the switch 236 to reconfigure the switch 236 into the connected configuration and turn on the lights 230. The lights 230 emit the activation light toward the dish 204 and the tissue sample 208. A user may then evaluate if any visible light is emitted by the tissue sample 208 to determine if any positive margins are present on the tissue sample 208.

As utilized herein with respect to numerical ranges, the terms “approximately,” “about,” “substantially,” and similar terms generally mean+/−10% of the disclosed values. When the terms “approximately,” “about,” “substantially,” and similar terms are applied to a structural feature (e.g., to describe its shape, size, orientation, direction, etc.), these terms are meant to cover minor variations in structure that may result from, for example, the manufacturing or assembly process and are intended to have a broad meaning in harmony with the common and accepted usage by those of ordinary skill in the art to which the subject matter of this disclosure pertains. Accordingly, these terms should be interpreted as indicating that insubstantial or inconsequential modifications or alterations of the subject matter described and claimed are considered to be within the scope of the disclosure as recited in the appended claims.

It should be noted that the term “exemplary” and variations thereof, as used herein to describe various embodiments, are intended to indicate that such embodiments are possible examples, representations, or illustrations of possible embodiments (and such terms are not intended to connote that such embodiments are necessarily extraordinary or superlative examples).

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

References herein to the positions of elements (e.g., “top,” “bottom,” “above,” “below”) are merely used to describe the orientation of various elements in the FIGURES. It should be noted that the orientation of various elements may differ according to other exemplary embodiments, and that such variations are intended to be encompassed by the present disclosure.

The hardware and data processing components used to implement the various processes, operations, illustrative logics, logical blocks, modules and circuits described in connection with the embodiments disclosed herein may be implemented or performed with a general purpose single- or multi-chip processor, a digital signal processor (DSP), an application specific integrated circuit (ASIC), a field programmable gate array (FPGA), or other programmable logic device, discrete gate or transistor logic, discrete hardware components, or any combination thereof designed to perform the functions described herein. A general purpose processor may be a microprocessor, or, any conventional processor, controller, microcontroller, or state machine. A processor also may be implemented as a combination of computing devices, such as a combination of a DSP and a microprocessor, a plurality of microprocessors, one or more microprocessors in conjunction with a DSP core, or any other such configuration. In some embodiments, particular processes and methods may be performed by circuitry that is specific to a given function. The memory (e.g., memory, memory unit, storage device) may include one or more devices (e.g., RAM, ROM, Flash memory, hard disk storage) for storing data and/or computer code for completing or facilitating the various processes, layers and modules described in the present disclosure. The memory may be or include volatile memory or non-volatile memory, and may include database components, object code components, script components, or any other type of information structure for supporting the various activities and information structures described in the present disclosure. According to an exemplary embodiment, the memory is communicably connected to the processor via a processing circuit and includes computer code for executing (e.g., by the processing circuit or the processor) the one or more processes described herein.

The present disclosure contemplates methods, systems and program products on any machine-readable media for accomplishing various operations. The embodiments of the present disclosure may be implemented using existing computer processors, or by a special purpose computer processor for an appropriate system, incorporated for this or another purpose, or by a hardwired system. Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. Combinations of the above are also included within the scope of machine-readable media. Machine-executable instructions include, for example, instructions and data which cause a general purpose computer, special purpose computer, or special purpose processing machines to perform a certain function or group of functions.

Although the figures and description may illustrate a specific order of method steps, the order of such steps may differ from what is depicted and described, unless specified differently above. Also, two or more steps may be performed concurrently or with partial concurrence, unless specified differently above. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

It is important to note that the construction and arrangement of the inspection assembly 200 as shown in the various exemplary embodiments is illustrative only. Additionally, any element disclosed in one embodiment may be incorporated or utilized with any other embodiment disclosed herein. For example, the camera 100, the controller 110, and the user interface 120 of the exemplary embodiment shown in at least FIG. 4 may be incorporated in the inspection assembly 200 of the exemplary embodiment shown in at least FIG. 6. Although only one example of an element from one embodiment that can be incorporated or utilized in another embodiment has been described above, it should be appreciated that other elements of the various embodiments may be incorporated or utilized with any of the other embodiments disclosed herein.

What is claimed is:

1. A surgical system for identifying cancerous tissue, the surgical system comprising:
a dish configured to receive a tissue sample;
a frame including a base defining a recess configured to receive the dish, an upright extending upward from the base, and an overhang extending over the base; and
a light source coupled to the overhang and positioned to emit light toward the recess.

2. The surgical system of claim 1, wherein the light source is configured to emit ultraviolet light.

3. The surgical system of claim 2, further comprising a container of cancer identifying fluid coupled to the frame, the cancer identifying fluid being configured to emit light in response to the cancer identifying fluid both (a) contacting the cancerous tissue along an exterior surface of the tissue sample and (b) being exposed to the ultraviolet light.

4. The surgical system of claim 3, wherein the cancer identifying fluid is configured to emit visible light in response to the cancer identifying fluid both (a) contacting the cancerous tissue along the exterior surface of the tissue sample and (b) being exposed to the ultraviolet light.

5. The surgical system of claim 1, wherein the light source is a first light source, further comprising a second light source coupled to the overhang and positioned to emit light toward the recess.

6. The surgical system of claim 5, wherein the base at least partially defines an internal volume of the frame, further comprising a battery electrically coupled to the first light source and the second light source and positioned within the internal volume.

7. The surgical system of claim 6, further comprising a switch coupled to the frame and configured to selectively electrically decouple the first light source and the second light source from the battery.

8. The surgical system of claim 1, further comprising a battery and a wire electrically coupling the battery to the light source, wherein the base, the upright, and the overhang at least partially define a passage, and wherein the wire passes through the passage.

9. The surgical system of claim 8, wherein the wire is a first wire, further comprising a switch coupled to the frame and a second wire electrically coupling the switch to the light source, wherein the first wire electrically couples the battery to the switch.

10. The surgical system of claim 1, wherein the base extends forward from the upright, and wherein the overhang extends forward from the upright.

11. A surgical method, comprising:
removing tissue from a patient, the tissue containing cancerous cells and having a margin that is exposed when the tissue is removed from the patient;
bathing the tissue in a cancer identifying fluid that is configured to emit light in response to the cancer identifying fluid both (a) contacting the cancerous cells and (b) being exposed to an activation light;
exposing the cancer identifying fluid to the activation light; and
determining if the cancerous cells are exposed along the margin based on whether or not the light is emitted by the cancer identifying fluid.

12. The surgical method of claim 11, wherein the cancer identifying fluid is configured to emit visible light in response to the cancer identifying fluid both (a) contacting the cancerous cells and (b) being exposed to the activation light.

13. The surgical method of claim 12, wherein the activation light is not visible.

14. The surgical method of claim 12, wherein the activation light includes ultraviolet light.

15. The surgical method of claim 11, wherein the tissue is first tissue, further comprising:
in response to a determination that the cancerous cells are exposed along the margin, removing second tissue from the patient.

16. The surgical method of claim 15, wherein the second tissue was previously in contact with the cancerous cells exposed along the margin.

17. The surgical method of claim 11, further comprising:
placing the tissue in a container supported by a frame, wherein the frame is coupled to a light source that emits the activation light.

18. The surgical method of claim 17, wherein the light source is positioned to direct the activation light downward toward the container.

19. The surgical method of claim 18, further comprising:
providing a battery coupled to the frame; and
supplying electrical energy from the battery to the light source.

20. A surgical system for identifying cancerous tissue, the surgical system comprising:
a dish configured to receive a tissue sample;
a frame including:
a first portion defining a recess configured to receive the dish;
a second portion extending above the first portion; and
a third portion extending between the first portion and the second portion and fixedly coupling the first portion to the second portion, the first portion, the second portion, and the third portion defining a passage;
a light source coupled to the third portion and positioned to emit ultraviolet light downward toward the dish;
a battery coupled to the frame and configured to supply electrical energy to the light source;
a wire extending through the passage and electrically coupling the battery and the light source; and
a container of cancer identifying fluid coupled to the frame, the cancer identifying fluid being configured to emit visible light in response to the cancer identifying fluid both (a) contacting the cancerous tissue and (b) being exposed to the ultraviolet light.
